(19) United States
(12) Patent Application Publication
(10) Pub. No.: US 2021/0174909 A1
(43) Pub. Date: Jun. 10, 2021

(54) GENERATIVE MACHINE LEARNING MODELS FOR PREDICTING FUNCTIONAL PROTEIN SEQUENCES

(71) Applicant: Homodeus, Inc., Guilford, CT (US)

(72) Inventors: Jonathan M. Rothberg, Guilford, CT (US); Zhizhuo Zhang, Branford, CT (US); Spencer Glantz, West Hartford, CT (US)

(73) Assignee: Homodeus, Inc., Guilford, CT (US)

(21) Appl. No.: 17/118,447

(22) Filed: Dec. 10, 2020

Related U.S. Application Data
(60) Provisional application No. 62/946,372, filed on Dec. 10, 2019.

(51) Int. Cl. G16B 40/00; G16B 35/10; C12N 15/10

(52) U.S. Cl. CPC G16B 40/00; C12N 15/1058; G16B 35/10

(57) ABSTRACT

The present disclosure provides, in some embodiments, techniques for using generative machine learning models to generate new functional protein sequences based on an input protein structure, such that the new functional protein sequences are structurally similar to the input protein structure but have new and diverse protein sequences. The techniques described herein may be used alone, or in conjunction with structural prediction algorithms and/or to generate diversified gene libraries in directed evolution techniques.

### RELATED APPLICATION

This application claims the benefit under 35 U.S.C. § 119(e) of U.S. provisional application No. 62/946,372, filed Dec. 10, 2019, which is incorporated by reference herein in its entirety.

### BACKGROUND

Proteins are macromolecules that are comprised of strings of amino acids, which interact with each other and fold into complex three-dimensional shapes with characteristic structures.

### SUMMARY

Provided herein, in some aspects, are methods for training a generative machine learning model to generate multiple candidate protein sequences, wherein the multiple candidate protein sequences may have protein structures similar to an input protein structure, and wherein the multiple candidate protein sequences differ from a set of known protein sequences having protein structures similar to the input protein structure.

According to one aspect, a system for generating multiple diverse candidate protein sequences based on an input protein structure is provided, wherein the system may comprise: at least one hardware processor; and at least one non-transitory computer-readable storage medium storing processor-executable instructions that, when executed by the at least one hardware processor, cause the at least one hardware processor to perform: receiving the input protein structure; accessing a set of known protein sequences having protein structures similar to the input protein structure; accessing a generative machine learning model configured to generate a candidate protein sequence upon receiving a protein structure as input; and generating multiple diverse candidate protein sequences by repeatedly: providing the input protein structure to the generative machine learning model as input, in order to generate a resulting candidate protein sequence; conditionally determining whether to include or exclude the resulting candidate protein sequence from the multiple diverse candidate protein sequences, based at least on a metric of similarity between the resulting candidate protein sequence and the set of known protein sequences.

In some embodiments, conditionally determining whether to include or exclude the resulting candidate protein sequence may comprise determining to exclude the resulting candidate protein sequence if the metric of similarity between the resulting candidate protein sequence and the set of known protein sequences is above a threshold.

In some embodiments, the metric of similarity may be an identity percentage.

In some embodiments, the set of known protein sequences having protein structures similar to the input protein structure may comprise protein sequences having protein structures with a root-mean-square deviation from the input protein structure below a threshold.

In some embodiments, generating multiple diverse candidate protein sequences may be repeated until a set number of diverse candidate protein sequences are generated.

In some embodiments, the input protein structure may be an experimentally-determined protein structure.

In some embodiments, the input protein structure may be an output of a structural prediction algorithm.

According to one aspect, a method of training a generative machine learning model to generate multiple candidate protein sequences, wherein at least one protein sequence of the multiple candidate protein sequences has a protein structure similar to a primary input protein structure, and wherein the at least one protein sequence differs from a set of known protein sequences having protein structures similar to the primary input protein structure, is provided. The method may comprise using computer hardware to perform: accessing a plurality of target protein sequences, wherein each target protein sequence of the plurality of target protein sequences represents a target training output of the generative machine learning model; accessing a plurality of input protein structures, wherein each input protein structure of the plurality of input protein structures corresponds to a target protein sequence of the plurality of target protein sequences and represents an input to the generative machine learning model for a corresponding target training output; and training the generative machine learning model using the plurality of target protein sequences and the plurality of input protein structures, to obtain the trained generative machine learning model.

In some embodiments, the method may further comprise using computer hardware to perform: accessing the primary input protein structure; providing the primary input protein structure as input to the trained generative machine learning model; and generating the multiple candidate protein sequences.

In some embodiments, the method may further comprise using computer hardware to perform: based on the multiple candidate protein sequences, producing a library of protein sequences for use in a directed protein evolution process.

In some embodiments, the method may further comprise using computer hardware to perform: filtering the multiple candidate protein sequences, wherein filtering the multiple candidate protein sequences comprises: determining a metric of similarity between a candidate protein sequence of the multiple candidate protein sequences and a known protein sequence of the set of known protein sequences having protein structures similar to the primary input protein structure; and conditionally excluding the candidate protein sequence from the multiple candidate protein sequences based on the determined metric of similarity.

In some embodiments, conditionally excluding the candidate protein sequence from the multiple candidate protein sequences based on the determined metric of similarity may comprise: excluding the candidate protein sequence if the determined metric of similarity is above a threshold.

In some embodiments, filtering the multiple candidate protein sequences may be performed repeatedly in conjunction with generating the multiple candidate protein sequences.

In some embodiments, filtering the multiple candidate protein sequences may be performed repeatedly in conjunction with generating the multiple candidate protein sequences, until a count of the multiple candidate protein sequences is above a threshold.

In some embodiments, the generative machine learning model may comprise: an encoding phase; a sampling phase; and a decoding phase.

In some embodiments, the encoding phase and decoding phase may utilize one or more residual networks.

In some embodiments, the primary input protein structure and the plurality of input structures may comprise information representing a three-dimensional protein backbone structure.

In some embodiments, the information representing the three-dimensional protein backbone structure may be a list of torsion angles.

According to one aspect, a method for performing directed evolution of proteins is provided, the method comprising iteratively performing: producing a library of protein sequences based on an input protein structure, using a generative machine learning model configured to generate protein sequences having protein structures similar to an input protein structure; expressing the protein sequences of the library of protein sequences; selecting and amplifying at least a portion of the expressed protein sequences; providing the selected and amplified protein sequences as input to a protein structure prediction algorithm configured to output a predicted protein structure.

In some embodiments, the input protein structure may have a desired function.

The foregoing summary is provided by way of illustration and is not intended to be limiting.

### BRIEF DESCRIPTION OF THE DRAWINGS

FIG. 1 is flow diagram of an illustrative process for generating new functional protein sequences.

FIG. 2 is a flow diagram illustrating a machine-learning guided platform for directed evolution.

FIG. 3 a flow diagram illustrating an exemplary implementation of a generative machine learning model according to the techniques described herein.

FIG. 4 is a flow diagram illustrating an exemplary ResBlock, according to some embodiments of the techniques described herein.

FIG. 5 is a sketch illustrating pseudo code for generating diverse (“low-identity”) functional protein sequences, according to some embodiments.

FIG. 6 is a block diagram of an illustrative implementation of a computer system for generating functional protein sequences based on protein structures.

### DETAILED DESCRIPTION

Proteins are biological machines with many industrial and medical applications; proteins are used in detergents, cosmetics, bioremediation, the catalysis of industrial-scale reactions, life science research, agriculture, and the pharmaceutical industry, with many modern drugs derived from engineered recombinant proteins. Generating new functional proteins, which exhibit increased function with respect to some desired activity, can be a fundamental step in engineering proteins for a variety of practical applications such as these. The fitness of a protein with respect to a particular function may be closely related to the three-dimensional (3D) structure of that protein.

Directed evolution is one process by which new functional proteins may be generated. In the context of functional protein generation, directed evolution may involve a repeated process of diversifying, selecting, and amplifying proteins over time. In general, such a process may begin with a diversified gene library, from which proteins may be expressed and then selected based on their fitness with respect to a desired function. The selected proteins may then be sequenced, and the corresponding genetic sequences amplified in order to be diversified for the next cycle of selection and amplification.

As proteins are repeatedly selected based on their fitness with respect to a desired function, increasingly fit protein variants are incrementally generated over time. Directed evolution may be thought of as traversing a local protein function fitness landscape, wherein the rounds of selection determine the most optimal gradient in the protein function fitness landscape given the starting point of the initial diversified gene library. Applicants have recognized and appreciated that having a better designed initial diversified gene library results in a better exploration of the protein function fitness landscape, thereby minimizing the number of rounds of evolution required to converge to an optimum and providing a resulting reduction of the cost and time associated with generating functional proteins. Thus, as described herein, designing initial diversified gene libraries with enhanced properties, such as increased diversity or greater initial protein function fitness, is advantageous for the directed evolution of functional proteins.

Despite the importance of the design of the initial diversified gene library, Applicants have recognized that traditional methods for generating diversified gene libraries are far from optimal. Random mutagenesis, one common approach for generating diversified gene libraries, results in randomized mutagenesis of a genetic sequence without regard to the structural or functional importance of sequence motifs within the genetic sequences. Thus, as appreciated by Applicants, diversified gene libraries produced with random mutagenesis therefore consist mostly of non-functional sequences; a small fraction of the library may be functional, and only a few variants (if any at all) may exhibit increased function with respect to the desired activity. Furthermore, random mutagenesis does not take into account cooperative relationships among amino acid residues—whereby mutation at one position may necessitate one or more compensatory mutations at other positions to maintain a given structure/function.

Applicants have further recognized and appreciated that targeted mutagenesis—the rational selection of positions to mutate in a genetic library—may be an alternative to random mutagenesis. However, targeted mutagenesis relies on the rational guidance of a protein designer, and among other limitations, cannot be used to widely explore a protein function fitness landscape, which may have many local minima and many non-obvious sequences with high fitness. In some cases, artificial intelligence may be integrated with techniques such as in targeted mutagenesis. For example, protein structure prediction algorithms may be trained on protein sequences with known, experimentally-derived structures, allowing ab initio structure predictions for new sequences. These structures may be useful for guiding a protein designer in the rational design of diversified gene libraries, but still require manual effort on the part of a protein designer. Given the limitations of random mutagenesis, targeted mutagenesis, and other diversification strategies, including, alternatively or additionally, DNA shuffling and chimera-genesis, Applicants had an interest in developing improved techniques for the design of diversified gene libraries.

Applicants have discovered and appreciated that computational models may be leveraged not just to predict structural aids for human designers, as described above, but also to design new functional protein sequences, such as may be used in the context of generating diversified gene libraries for directed evolution. One method for functional sequence design, as Applicants have appreciated, is to start with the known protein backbone structure of a functional protein, and to use physics-based modeling to determine the set of allowable amino acids substitutions that would not result in large scale structural disruption but could permit new or enhanced function. This approach relies on physics-based computational modeling tools to perform comprehensive side-chain sampling on the known protein backbone structure to determine which amino acid substitutions and in which side-chain conformation would still permit the 3D folding of the functional protein.

Applicants have further discovered that non-physics based, machine-guided approaches to new functional protein design may be especially advantageous in the context of generating diversified gene libraries. For example, generative machine learning models, which are machine learning models that learn to represent the statistics of their input distributions as a joint probability distribution, may be employed to generate new function protein sequences. Examples of generative models include autoencoders, variational autoencoders (VAEs), and generative adversarial networks (GANs). Autoencoder machine learning models learn to encode an input sequence in lower dimensional space (a vector), called the latent space, and decode the latent-space vector to reconstruct the input.

Traditionally, generative machine learning models for generating new functional protein sequences may learn to encode protein sequences into a latent space in which distances are meaningful, mapping similar proteins to nearby points in latent space. Generative models can be trained, for example, on libraries of known functional sequences from a given protein family or set of families and can learn the distribution of mutations that preserve function or family identity. The benefit of using deep-learning based generative models to represent the distribution of protein sequences in a given family is that these models can learn higher-order correlations beyond the pairwise residue correlations captured by other models such as Canonical Correlation Analysis (CCA) and Direct Coupling Analysis (DCA). These generative models, once trained, may then be used to produce new protein sequences that have not been observed in nature, but are likely to be functional members of the protein family that the generative model was trained on. Applicants have also recognized and appreciated that generative models for generating new functional protein sequences may be trained on protein structures. In such cases, the 3D protein structure may be encoded in low dimensional space, and a decoder network may be used generatively to predict homologous functional protein sequences that would fold into the desired structure.

The present disclosure provides, according to some embodiments described herein, a generative machine learning model that generates new functional protein sequences given an input protein structure, yielding multiple candidate protein sequences that are diverse (e.g. different in sequence from known, natural protein sequences) yet are likely to retain a same or similar 3D structure to the input protein structure.

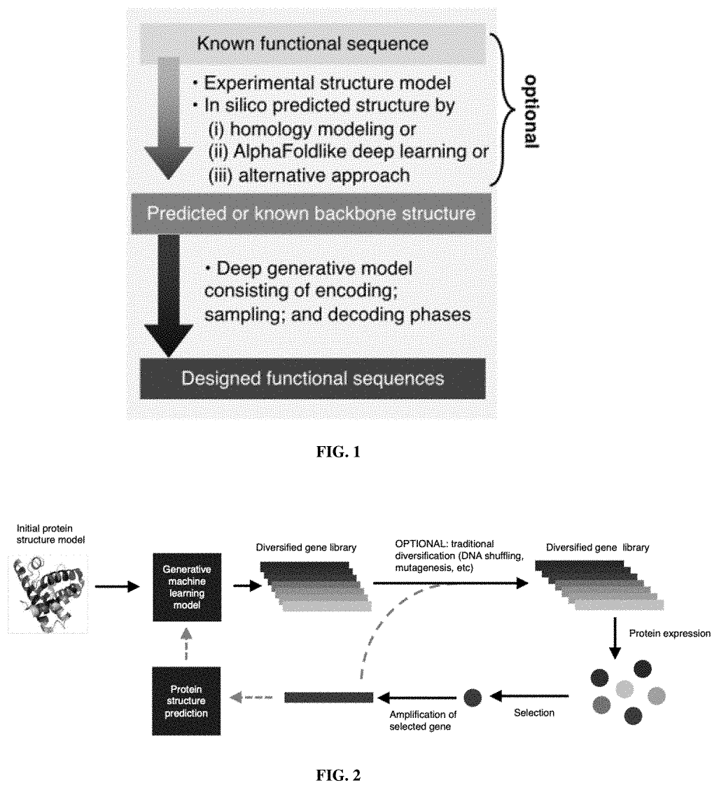

FIG. 1 is flow diagram of an illustrative process for generating new functional protein sequences according to some of the techniques described herein. As shown in the illustrated example, the input protein structure may be an experimentally-derived (e.g. known) structure model. In other examples, the protein structure provided as input to a generative machine learning model may itself optionally be an output of an in silico protein structure prediction algorithm. In silico protein structure prediction algorithms may include, for example, homology modeling, modeling with machine learning, or alternative approaches.

Regardless of how the input protein structure is derived, it may then serve as an input to generative machine learning model, as shown in the figure. In the illustrated example, the input protein structure is a backbone structure of the protein. The backbone structure of the protein may be indicative of the overall structure of the protein and may be represented as a list of Cartesian coordinates of protein backbone atoms (alpha-carbon, beta-carbon and N terminal) or a list of torsion angles of the protein backbone structure. Regardless of how the input protein structure is represented, the generative machine learning model may process the input protein structure in phases of encoding, sampling, and decoding, as indicated in the figure, and described in detail below, in order to produce as output new functional protein sequences.

According to some embodiments, a generative machine learning model such as the one described with reference to FIG. 1 may be used alone, or iteratively in conjunction with an in silico protein structure prediction algorithm to allow for a closed-loop, machine-learning guided platform for directed evolution. FIG. 2 is a flow diagram illustrative of such a closed-loop, machine-learning guided platform for directed evolution, such as may be used to design new functional protein sequences having enhanced or optimal fitness with respect to a desired function. As shown in the illustrated example, a directed evolution process using a generative machine learning model according to the techniques described herein may involve the following steps:

(i) an initial protein structure model is provided as the input protein structure to a generative machine learning model, such as described above;

(ii) the generative machine learning model generates new protein sequences predicted to fold into the input protein structure;

(iii) a diversified gene library is synthesized from the new protein sequences

(iv) optionally, the gene library may be further diversified, for example by mutagenesis or DNA shuffling or other suitable techniques;

(v) the diversified gene library is expressed;

(vi) high fitness proteins are selected from the expressed proteins;

(vii) the selected proteins are sequenced, and the genes coding for the selected proteins are amplified;

(viii) the amplified gene sequences are diversified for another cycle of selection and amplification. Diversification may be achieved by:
1. repeating steps (iv)-(vii).
2. the amplified gene sequences are fed into a protein structure prediction algorithm; and then steps (ii)-(vii) are repeated.

This completes the closed-loop cycle of directed evolution, which may be run iteratively as protein sequences converge on a functional protein sequence with optimal fitness with respect to a desired function. It should be appreciated that some steps of the process illustrated in FIG. 2 are optional and may be skipped or replaced with alternative steps in some embodiments. For example, the use of traditional diversification techniques in (iii) need not take place in every iteration and may not take place in any iterations. It should also be appreciated that the process illustrated in FIG. 2 need not repeat ad infinitum, but may instead terminate, such as when the protein sequences have converged on a functional protein sequence with a degree of fitness with respect to a desired function above a threshold.

In the context of a closed loop directed evolution cycle, as shown in FIG. 2, the generative machine learning model serves to produce a higher quality diversified gene library than may be obtained by random mutagenesis or other traditional techniques. Having learned the distribution of sequences that fold to structures similar to the input structure, as described in detail below, the generative machine learning model produces multiple candidate protein sequences for inclusion in the diversified gene library that are significantly more likely to fold and function similarly to, or better than, the original input sequence, when compared to candidates sequences obtained through random mutagenesis or other traditional techniques. Moreover, although the space of possible protein sequences of a given length is astronomically large, the generative machine learning model learns to only produce sequences that are likely to have a similar functionality and structure as a given target.

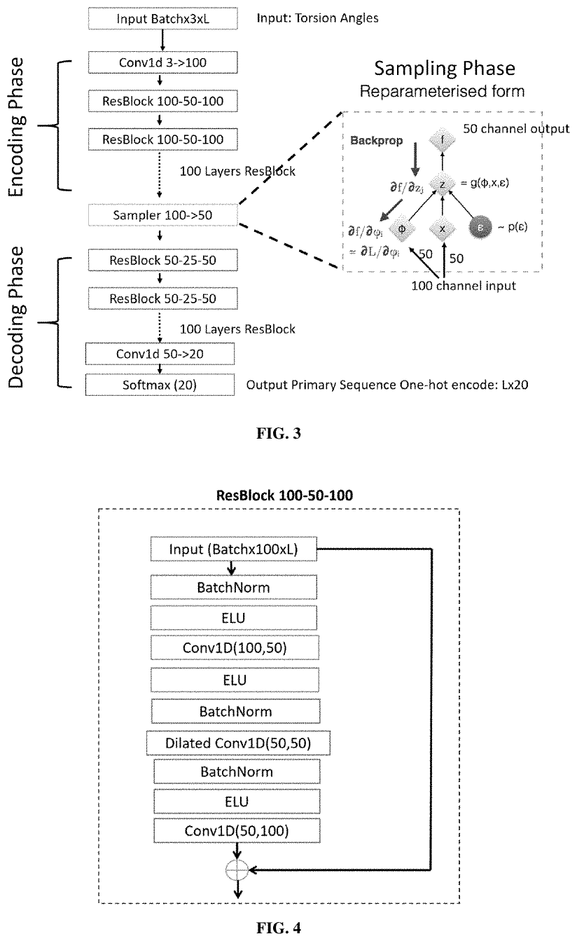

In FIG. 3, a flow diagram illustrating an exemplary implementation of a generative machine learning model according to the techniques described herein is provided. In the illustrated example, the generative machine learning is implemented as a deep neural network comprising phases of encoding, sampling, and decoding. It should be appreciated that the deep neural network of FIG. 3 is exemplary, and that alternative machine learning methods and architectures may be employed in some embodiments of the techniques described herein.

The deep neural network of FIG. 3 may be configured to generate multiple candidate protein sequences given an input protein 3D backbone structure. The 3D backbone structure could be represented by Cartesian coordinates of protein backbone atoms (alpha-carbon, beta-carbon and N terminal) or list of torsion angles of the protein backbone structure, as described above with reference to FIG. 1. Cartesian coordinates of protein backbone atoms can be directly converted to a sequence of triplet dihedral angles (Ω, Ψ, Φ), hence, the deep neural network of FIG. 3 inputs of list of torsion angles according to this format. For a protein structure with L amino acid residues, the protein structure could thus be represented by L×3 matrix, that is, 3 torsion angles (Ω, Ψ, Φ), for each amino acid residue.

In the illustrated example, the model consists of three phases, which may proceed as described in the following:
1. Encoding phase: The input layer is propagated through a one-dimensional convolution (Conv1D), which projects from 3 dimensions to 100 dimensions in order to generate a 100×L matrix. This matrix is iterated 100 times through residual network (RESNET) blocks (see FIG. 4, showing an exemplary ResBlock) which perform batch normalizing, apply an exponential linear unit (ELU) activation function, project down to a 50×L matrix, apply batch normalizing and ELU again, and then cycle through 4 different dilation filters. The dilation filters have sizes 1,2,4, and 8 and are applied with a padding of the same to retain dimensionality. A final batch normalization is performed, then the matrix is projected up to 100×L and an identity addition is performed.
2. Sampling phase: A 100×L matrix is generated from the encoding phase, and the first 50 dimensions from the encoded vector in each position serve as the mean of 50 Gaussian distributions, while the last 50 dimensions serve the corresponding log of variance of those Gaussian distributions. Applying reparameterization, the model samples the hidden variable z from the 50 Gaussian distributions, which together generates a 50×L matrix as output from the sampling phase.
3. Decoding phase: The decoding phase input is the 50×L matrix output from the sampling phase, and it is iterated 100 times through ResBlocks similar to those in the encoding phase (see FIG. 4). Here, however, the ResBlocks map 50 input dimensions to 50 output dimensions. After the ResBlock layers, the model reshapes the 50 dimensions to 20 dimensions (corresponding to 20 amino acids) using a one-dimensional convolution with kernel size 1 and applies softmax to the 20 dimensions. The final output matrix dimension is 20×L, which presents the probability of 20 amino acid in each residue position.

FIG. 4 is a flow diagram illustrating an exemplary ResBlock, according to some embodiments of the techniques described herein. As was described with reference to FIG. 3, this flow diagram indicates that a ResBlock may function according to the following steps:
(i) Applies batch normalizing (BatchNorm);
(ii) Applies the exponential linear unit (ELU) activation function;
(iii) Projects down to a 50×L matrix using a one-dimensional convolution (Conv1D);
(iv) Applies batch normalizing (BatchNorm) and ELU;
(v) Cycles through 4 different dilation filters (Dilated Conv1D), having sizes 1,2,4, and 8 a padding of the same to retain dimensionality;
(vi) Applies batch normalizing, projecting the matrix up to 100×L;
(vii) Performs an identity addition.

It is envisioned that steps of any of the methods described herein can be encoded in software and carried out by a processor, such as that of a general purpose computer, when implementing the software. Some software algorithms envisioned may include artificial intelligence based machine learning algorithms, trained on an initial set of data, and improved as the data increases.

A deep neural network according to the techniques described herein, such as illustrated in FIGS. 3 and 4, for example, may be trained by providing training data to the network in pairs of input protein structures and corresponding target protein sequences. In order to learn a statistical model of the input distribution, an input protein structure may be provided as input to the deep neural network, which may output a protein sequence, such as by the process described with respect to FIGS. 3 and 4 above. A loss value may then be calculated between the neural network's output protein sequence, and the target protein sequence corresponding to the input protein structure. Then, a gradient descent optimization method can be applied to update weights or other parameters of the neural network such that the loss value is minimized.

As a specific example of training, such a deep neural network may be trained using existing protein/domain structure databases like PDB (Protein Data Bank) and CATH (Class, Architecture, Topology, Homologous superfamily), which contain both structure and primary sequence information. The information of given backbone structure may firstly be converted to a list of torsion angles. The list of torsion angles may be provided as input to the neural network, which may output a 20 dimension probability vector for each residue, representing the probability of 20 amino acid in each residue position. A cross-entropy loss may be computed between the output probability vectors and true primary sequence; then, any general stochastic gradient descent optimization method can be applied to update the model parameters and minimize the loss value.

It should be appreciated that any of the parameters of a deep neural network according to the techniques described herein may differ from those in the example of FIGS. 3 and 4. For example, in some embodiments, the dimensionality of the layers of the deep neural network may differ, or other parameters that may be associated with the network, such as type and number of activation functions, loss function, learning rate, optimization function, etc., may be adjusted. Moreover, the architecture of the deep neural network may differ in some embodiments. For example, differing layer types may be employed, and techniques such as layer dropout, pooling, or normalization may be applied.

With regards to the techniques described herein for generating new functional protein sequences, Applicants have further discovered and appreciated that in order to generate enhanced diversified gene libraries, it is not only important that functional protein sequences are generated that could fold into a given input protein structure (so as to retain some degree of function), but also that the generated functional protein sequences are diverse—that is, they are dissimilar to the set of known or naturally-occurring sequences associated with the input protein structure. New functional proteins generated in such a way are more likely to have new or enhanced function, relative to functional proteins generated by traditional methods, and thus provide an initial diversified gene library with increased diversity and protein function fitness.

According to some embodiments, new functional protein sequences that exhibit increased diversity with respect to an input protein structure may be generated by first determining a set of known protein sequences having a structure similar to the input protein structure, then repeatedly generating candidate functional protein sequences and discarding any that are determined to be too similar to members of the set of known protein sequences. As part of repeatedly generating candidate functional protein sequences, a generative machine learning model, such as according to the techniques described herein, may be employed.

As a specific example, new functional protein sequences that exhibit increased diversity may be produced by the following method:

1. Given an input protein structure (e.g. only consider the backbone), search all similar structures (e.g. could be domain structure) under certain similarity criteria (e.g. Root-mean-square deviation below a certain threshold, such as 2), and obtain the primary sequences for those similar structures as the set of known sequences that fold into those structures.

2. Use a generative model, such as one according to the techniques described herein, to generate new functional protein sequences from the given input structure. Accept the generated sequence only if the generated sequence is below a certain similarity threshold (e.g. identity percentage less than a threshold, such as 80%) to all the sequences in the set of known sequences. The generative model would stop once the number of accepted sequences reaches a specified value (e.g. specified by a user).

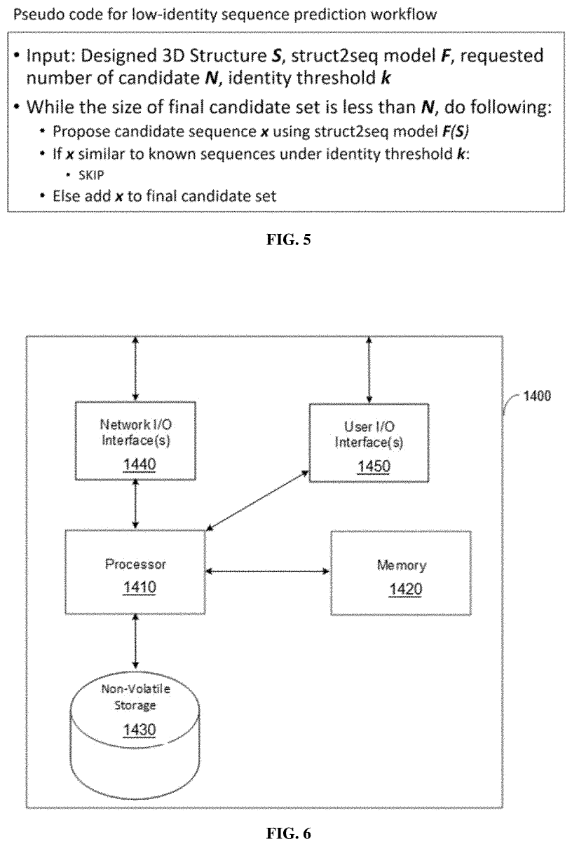

FIG. 5 is a diagram illustrating pseudo code for generating diverse (“low-identity”) functional protein sequences, according to some embodiments. As input, the pseudo code takes in a 3D Structure S (e.g. a protein structure, represented in any suitable way), a struct2seq model F (e.g. any suitable generative machine learning model), a requested number of candidate N (e.g. the desired number of new functional protein sequences), and an identity threshold k (e.g. an upper bound on the allowable similarity between a generated functional protein sequence, and known sequences). As described above, the pseudo code then enters a loop wherein a final candidate set is populated by means of repeatedly: proposing a candidate sequence x using F(S); checking if x is similar to known sequences under k; skipping x if so, and adding x to the final candidate set otherwise. This process is repeated until the size of the final candidate set is equal to N, at which point the process ends.

An illustrative implementation of a computer system 1400 that may be used in connection with any of the embodiments of the technology described herein is shown in FIG. 6. The computer system 1400 includes one or more processors 1410 and one or more articles of manufacture that comprise non-transitory computer-readable storage media (e.g., memory 1420 and one or more non-volatile storage media 1430). The processor 1410 may control writing data to and reading data from the memory 1420 and the non-volatile storage device 1430 in any suitable manner, as the aspects of the technology described herein are not limited in this respect. To perform any of the functionality described herein, the processor 1410 may execute one or more processor-executable instructions stored in one or more non-transitory computer-readable storage media (e.g., the memory 1420), which may serve as non-transitory computer-readable storage media storing processor-executable instructions for execution by the processor 1410.

Computing device 1400 may also include a network input/output (I/O) interface 1440 via which the computing device may communicate with other computing devices (e.g., over a network), and may also include one or more user I/O interfaces 1450, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

The above-described embodiments can be implemented in any of numerous ways. For example, the embodiments may be implemented using hardware, software or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-discussed functions of one or more embodiments. The computer-readable medium may be transportable such that the program stored thereon can be loaded onto any computing device to implement aspects of the techniques discussed herein. In addition, it should be appreciated that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

### Additional Embodiments

Additional embodiments of the present disclosure are encompassed by the following numbered paragraphs.

1. A system for generating multiple diverse candidate protein sequences based on an input protein structure, the system comprising:

at least one hardware processor; and

at least one non-transitory computer-readable storage medium storing processor-executable instructions that, when executed by the at least one hardware processor, cause the at least one hardware processor to perform:
receiving the input protein structure;
accessing a set of known protein sequences having protein structures similar to the input protein structure;
accessing a generative machine learning model configured to generate a candidate protein sequence upon receiving a protein structure as input; and
generating multiple diverse candidate protein sequences by repeatedly:
   providing the input protein structure to the generative machine learning model as input, in order to generate a resulting candidate protein sequence;
   conditionally determining whether to include or exclude the resulting candidate protein sequence from the multiple diverse candidate protein sequences, based at least on a metric of similarity between the resulting candidate protein sequence and the set of known protein sequences.

2. The system of paragraph 1, wherein conditionally determining whether to include or exclude the resulting candidate protein sequence comprises determining to exclude the resulting candidate protein sequence if the metric of similarity between the resulting candidate protein sequence and the set of known protein sequences is above a threshold.

3. The system of paragraph 1 or 2, wherein the metric of similarity is an identity percentage.

4. The system of any one of paragraphs 1-3, wherein the set of known protein sequences having protein structures similar to the input protein structure comprises protein sequences having protein structures with a root-mean-square deviation from the input protein structure below a threshold.

5. The system of any one of paragraphs 1-4, wherein the generating multiple diverse candidate protein sequences is repeated until a set number of diverse candidate protein sequences are generated.

6. The system of any one of paragraphs 1-5, wherein the input protein structure is an experimentally-determined protein structure.

7. The system of any one of paragraphs 1-6, wherein the input protein structure is an output of a structural prediction algorithm.

8. A method of training a generative machine learning model to generate multiple candidate protein sequences, wherein at least one protein sequence of the multiple candidate protein sequences has a protein structure similar to a primary input protein structure, and wherein the at least one protein sequence differs from a set of known protein sequences having protein structures similar to the primary input protein structure, the method comprising using computer hardware to perform:

accessing a plurality of target protein sequences, wherein each target protein sequence of the plurality of target protein sequences represents a target training output of the generative machine learning model;

accessing a plurality of input protein structures, wherein each input protein structure of the plurality of input protein structures corresponds to a target protein sequence of the plurality of target protein sequences and represents an input to the generative machine learning model for a corresponding target training output; and

training the generative machine learning model using the plurality of target protein sequences and the plurality of input protein structures, to obtain the trained generative machine learning model.

9. The method of paragraph 8, further comprising using computer hardware to perform:

accessing the primary input protein structure;

providing the primary input protein structure as input to the trained generative machine learning model; and

generating the multiple candidate protein sequences.

10. The method of paragraph 9, further comprising using computer hardware to perform:

based on the multiple candidate protein sequences, producing a library of protein sequences for use in a directed protein evolution process.

11. The method of paragraph 9, further comprising using computer hardware to perform:

filtering the multiple candidate protein sequences, wherein filtering the multiple candidate protein sequences comprises:
determining a metric of similarity between a candidate protein sequence of the multiple candidate protein sequences and a known protein sequence of the set of known protein sequences having protein structures similar to the primary input protein structure; and
conditionally excluding the candidate protein sequence from the multiple candidate protein sequences based on the determined metric of similarity.

12. The method of paragraph 11, wherein conditionally excluding the candidate protein sequence from the multiple candidate protein sequences based on the determined metric of similarity comprises:

excluding the candidate protein sequence if the determined metric of similarity is above a threshold.

13. The method of paragraph 11 or 12, wherein filtering the multiple candidate protein sequences is performed repeatedly in conjunction with generating the multiple candidate protein sequences.

14. The method of any one of paragraphs 11-13, wherein filtering the multiple candidate protein sequences is performed repeatedly in conjunction with generating the multiple candidate protein sequences, until a count of the multiple candidate protein sequences is above a threshold.

15. The method of any one of paragraphs 8-14, wherein the generative machine learning model comprises:

an encoding phase;

a sampling phase; and

a decoding phase.

16. The method of paragraph 15, wherein the encoding phase and decoding phase utilize one or more residual networks.

17. The method of any one of paragraphs 8-16, wherein the primary input protein structure and the plurality of input structures comprise information representing a three-dimensional protein backbone structure.

18. The method of paragraph 17, wherein the information representing the three-dimensional protein backbone structure is a list of torsion angles.

19. A method for performing directed evolution of proteins, the method comprising iteratively performing:

producing a library of protein sequences based on an input protein structure, using a generative machine learning model configured to generate protein sequences having protein structures similar to an input protein structure;

expressing the protein sequences of the library of protein sequences;

selecting and amplifying at least a portion of the expressed protein sequences;

providing the selected and amplified protein sequences as input to a protein structure prediction algorithm configured to output a predicted protein structure.

20. The method of paragraph 19, wherein the input protein structure has a desired function.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between the upper and lower ends of the range are specifically contemplated and described herein.

What is claimed is:

1. A system for generating multiple diverse candidate protein sequences based on an input protein structure, the system comprising:
at least one hardware processor; and
at least one non-transitory computer-readable storage medium storing processor-executable instructions that, when executed by the at least one hardware processor, cause the at least one hardware processor to perform:
receiving the input protein structure;
accessing a set of known protein sequences having protein structures similar to the input protein structure;
accessing a generative machine learning model configured to generate a candidate protein sequence upon receiving a protein structure as input; and
generating multiple diverse candidate protein sequences by repeatedly:
providing the input protein structure to the generative machine learning model as input, in order to generate a resulting candidate protein sequence;
conditionally determining whether to include or exclude the resulting candidate protein sequence from the multiple diverse candidate protein sequences, based at least on a metric of similarity between the resulting candidate protein sequence and the set of known protein sequences.

2. The system of claim 1, wherein conditionally determining whether to include or exclude the resulting candidate protein sequence comprises determining to exclude the resulting candidate protein sequence if the metric of similarity between the resulting candidate protein sequence and the set of known protein sequences is above a threshold.

3. The system of claim 1, wherein the metric of similarity is an identity percentage.

4. The system of claim 1, wherein the set of known protein sequences having protein structures similar to the input protein structure comprises protein sequences having protein structures with a root-mean-square deviation from the input protein structure below a threshold.

5. The system of claim 1, wherein the generating multiple diverse candidate protein sequences is repeated until a set number of diverse candidate protein sequences are generated.

6. The system of claim 1, wherein the input protein structure is an experimentally-determined protein structure.

7. The system of claim 1, wherein the input protein structure is an output of a structural prediction algorithm.

8. A method of training a generative machine learning model to generate multiple candidate protein sequences, wherein at least one protein sequence of the multiple candidate protein sequences has a protein structure similar to a primary input protein structure, and wherein the at least one protein sequence differs from a set of known protein sequences having protein structures similar to the primary input protein structure, the method comprising using computer hardware to perform:
accessing a plurality of target protein sequences, wherein each target protein sequence of the plurality of target protein sequences represents a target training output of the generative machine learning model;
accessing a plurality of input protein structures, wherein each input protein structure of the plurality of input protein structures corresponds to a target protein sequence of the plurality of target protein sequences and represents an input to the generative machine learning model for a corresponding target training output; and
training the generative machine learning model using the plurality of target protein sequences and the plurality of input protein structures, to obtain the trained generative machine learning model.

9. The method of claim 8, further comprising using computer hardware to perform:
accessing the primary input protein structure;
providing the primary input protein structure as input to the trained generative machine learning model; and
generating the multiple candidate protein sequences.

10. The method of claim 9, further comprising using computer hardware to perform:
based on the multiple candidate protein sequences, producing a library of protein sequences for use in a directed protein evolution process.

11. The method of claim 9, further comprising using computer hardware to perform:
filtering the multiple candidate protein sequences, wherein filtering the multiple candidate protein sequences comprises:
determining a metric of similarity between a candidate protein sequence of the multiple candidate protein sequences and a known protein sequence of the set of known protein sequences having protein structures similar to the primary input protein structure; and
conditionally excluding the candidate protein sequence from the multiple candidate protein sequences based on the determined metric of similarity.

12. The method of claim 11, wherein conditionally excluding the candidate protein sequence from the multiple candidate protein sequences based on the determined metric of similarity comprises:
excluding the candidate protein sequence if the determined metric of similarity is above a threshold.

13. The method of claim 11, wherein filtering the multiple candidate protein sequences is performed repeatedly in conjunction with generating the multiple candidate protein sequences.

14. The method of claim 11, wherein filtering the multiple candidate protein sequences is performed repeatedly in conjunction with generating the multiple candidate protein sequences, until a count of the multiple candidate protein sequences is above a threshold.

15. The method of claim 8, wherein the generative machine learning model comprises:
an encoding phase;
a sampling phase; and
a decoding phase.

16. The method of claim 15, wherein the encoding phase and decoding phase utilize one or more residual networks.

17. The method of claim 8, wherein the primary input protein structure and the plurality of input structures comprise information representing a three-dimensional protein backbone structure.

18. The method of claim 17, wherein the information representing the three-dimensional protein backbone structure is a list of torsion angles.

19. A method for performing directed evolution of proteins, the method comprising iteratively performing:
producing a library of protein sequences based on an input protein structure, using a generative machine learning model configured to generate protein sequences having protein structures similar to an input protein structure;
expressing the protein sequences of the library of protein sequences;
selecting and amplifying at least a portion of the expressed protein sequences;
providing the selected and amplified protein sequences as input to a protein structure prediction algorithm configured to output a predicted protein structure.

20. The method of claim 19, wherein the input protein structure has a desired function.
